(19) United States
(12) Patent Application Publication
(10) Pub. No.: US 2023/0134834 A1
(43) Pub. Date: May 4, 2023

(54) MECHANICAL INSUFFLATION-EXSUFFLATION DEVICE WITH ENHANCED DEVICE-PATIENT SYNCHRONIZATION AND METHOD OF OPERATION THEREOF

(71) Applicant: KONINKLIJKE PHILIPS N.V., Eindhoven (NL)

(72) Inventors: Kiran Hamilton J. Dellimore, Eindhoven (NL); David Aaron Troxell, Monroeville, PA (US); Privender Kaur Saini, Eindhoven (NL); Rita Priori, Eindhoven (NL); Seunghyun Lee, Monroeville, PA (US)

(21) Appl. No.: 17/903,184

(22) Filed: Sep. 6, 2022

Related U.S. Application Data
(60) Provisional application No. 63/273,191, filed on Oct. 29, 2021.

(51) Int. Cl. A61M 16/00; A61H 31/00

(52) U.S. Cl. CPC A61M 16/022; A61H 31/00; A61H 2230/405; A61H 2201/107; A61M 2016/0027; A61M 2016/0033; A61M 2202/0225; A61H 2205/084

(57) ABSTRACT

A mechanical insufflation-exsufflation device including an air source to provide positive airway pressure (PAP); a patient interface; at least one sensor to sense air pressure and flow at the patient interface; and a controller to control the air source to deliver at least one mechanically assisted cough to the patient in response to at least one of a target breathing flow and a target inhalation time period being sensed by the at least one sensor, and when the at least one of a target breathing flow and a target inhalation time period is not sensed, the controller is configured to control the air source to deliver each in a series of high-level PAP provided over a duration being followed by a low-level PAP provided over a duration, the series of high-level PAP increasing in pressure level and duration from a prior one in the series of high-level PAP.

### CROSS-REFERENCE TO RELATED APPLICATIONS

This patent application claims the priority benefit under 35 U.S.C. § 119(e) of U.S. Provisional Application No. 63/273,191, filed on Oct. 29, 2021, the contents of which are herein incorporated by reference.

### FIELD OF THE PRESENT SYSTEM

The present system relates to a mechanical insufflation-exsufflation (MI-E) device with enhanced device-patient synchrony during MI-E therapy and, more particularly, to a MI-E device which provides an improved device-patient synchrony during MI-E therapy based on passively timing delivery of mechanical insufflation using breath pacing, and a method of operation thereof.

### BACKGROUND OF THE PRESENT SYSTEM

A major challenge in the delivery of MI-E therapy to patients (e.g., a target patient group), who are either too young or incapable of following commands, is asynchrony between an MI-E device (e.g., a CoughAssist™ MI-E device by the Philips Healthcare) during insufflation and the patient's breathing pattern. This asynchrony may be referred to as device-patient asynchrony. This target patient group may, for example, hold their breath during either the insufflation or the exsufflation phase and/or exhale during the pause phase, which leads to ineffective therapy delivery.

Mechanical insufflation-exsufflation (MI-E) therapy has been described as safe and effective in the pediatric neuromuscular disease (NMD) patient population and is considered to be a critical component of the respiratory support strategy. In spite of this, it remains unclear how MI-E therapy can be optimized when a patient, such as a pediatric patient, is highly agitated and either too young or otherwise unable/unwilling to follow commands due to cognitive limitations, etc.

Effective MI-E therapy requires appropriate settings and is highly dependent on therapy technique. A key barrier to effective MI-E therapy is the inability to obtain adequate lung volume due to agitation and breath holding. Frequently, when pediatric patients are highly agitated and either too young or otherwise unable to follow commands, they may hold their breath during insufflation, they may hold their breath or inhale during exsufflation and/or may exhale during a pause phase, which leads to asynchrony between an MI-E device and the patient's breathing pattern, resulting in ineffective MI-E therapy delivery.

A key barrier to effective MI-E therapy appears to be the inability to obtain adequate lung volume due to: (a) agitation likely induced by the dishabituation to the negative and positive pressure sensation produced by the MI-E therapy; and/or (b) breath holding linked to lack of cooperation with the therapy.

Hence there is a need to overcome the aforementioned barriers, by finding novel ways to effectively deliver mechanically assisted coughs during MI-E therapy for patients including those who are too young or otherwise unable to follow commands. For example, embodiments of the present system address the problem of poor patient-device synchrony during the delivery of MI-E therapy to patients who are too young (i.e., infants) or unable to follow commands. More specifically, embodiments of the present system address the problem of how to synchronize mechanical insufflation with the breathing of a patient to ensure effective MI-E therapy delivery whether or not patient-device synchrony is initially present.

### SUMMARY OF THE PRESENT SYSTEM

The system(s), device(s), method(s), arrangements(s), interface(s), computer program(s), processes, etc., (hereinafter each of which will be referred to as system, unless the context indicates otherwise), described herein address problems in prior art systems.

In accordance with embodiments of the present system, there is disclosed a mechanical insufflation-exsufflation (MI-E) device. The device may include an air source configured to provide patient airway pressure including positive airway pressure (PAP); a patient interface coupled to the air source and configured to be flow coupled to a user; at least one sensor configured to sense air pressure and flow at the patient interface; and a controller coupled to the air source and the at least one sensor. The controller may be configured to control the air source to deliver at least one mechanically assisted cough (MAC) to the patient in response to at least one of a target breathing flow and a target inhalation time period being sensed by the at least one sensor. When the at least one of a target breathing flow and a target inhalation time period is not sensed by the at least one sensor, the controller may be configured to control the air source to deliver a series of high-level PAP, each in the series of high-level PAP provided over a high-level duration being followed by a low-level PAP provided over a low-level duration, the series of high-level PAP increasing in at least one of a pressure level and the high-level duration from a prior one in the series of high-level PAP. Each in the series of high-level PAP may start in response to a corresponding inhalation trigger. The series of low-level PAP may each have a pressure level that is lower than the pressure level of each of the series of high-level PAP.

The MI-E device may include a force transmitting device (FTD) coupled to the controller. The controller may be configured to control the FTD to apply an abdominal chest thrust that is synchronized to the MAC. The controller may be further be configured to receive flow and pressure information from the at least one sensor and generate corresponding flow and pressure waveforms. The controller may be further configured to produce each in the series of high-level PAP in response to detecting an inhalation trigger (TG) in a form of a change in pressure below ambient in the pressure waveform. The MI-E device may include a CO₂ controller coupled to the patient interface that may be configured to control a concentration of CO₂ gas at the patient interface.

The controller may be configured to control the air source to deliver the at least one mechanically assisted cough (MAC) to the patient when both of the target breathing flow and the target inhalation time period are sensed by the at least one sensor. The controller may be configured to determine whether the pressure level and the high-level duration of a current one of the series of the high-level PAP has a sensed pressure and a duration greater than, or equal to, a pressure threshold value and a high-level duration threshold value, respectively and may be configured to control the air source to provide a further one of the series of high-level PAP in response to the pressure level and the high-level duration of a current one of the series of steps of the high-level PAP being sensed to have a pressure and a duration that is not greater than, or equal to, the pressure threshold value and the high-level duration threshold value, respectively. The controller may be configured to increase the high-level duration of each of the series of high-level PAP by a predetermined period of time. The controller may be configured to increase the pressure of each of the series of high-level PAP by a predetermined pressure.

In accordance with embodiments of the present system, there is also disclosed a mechanical insufflation-exsufflation (MI-E) device including a controller that may be configured to control an air source to deliver at least one mechanically assisted cough (MAC) to a patient in response to at least one of a target breathing flow and a target inhalation time period being sensed by at least one sensor. When the at least one of a target breathing flow and a target inhalation time period is not sensed by the at least one sensor, the controller may also be configured to control the air source to deliver a series of high-level PAP, each in the series of high-level PAP provided over a high-level duration being followed by a low-level PAP provided over a low-level duration. The series of high-level PAP increasing in pressure level and duration from a prior one in the series of high-level PAP. Each in the series of high-level PAP starting in response to a corresponding inhalation trigger. The series of low-level PAP each having a pressure level that is lower than the pressure level of each of the series of high-level PAP.

The controller may be configured to control a force transmitting device (FTD) to apply an abdominal chest thrust that is synchronized to the MAC. The controller may be further configured to receive flow and pressure information from the at least one sensor and generate corresponding flow and pressure waveforms. The controller may be further configured to produce each in the series of high-level PAP in response to detecting an inhalation trigger (TG_{I}) in a form of a change in pressure below ambient in the pressure waveform. The controller may be further configured to control a concentration of CO₂ gas available to the patient. The controller may be configured to control the air source to deliver the at least one mechanically assisted cough (MAC) to the patient when both of the target breathing flow and the target inhalation time period are sensed by the at least one sensor.

The controller may be configured to determine whether the pressure level and the high-level duration of a current one of the series of the high-level PAP has a sensed pressure and a duration greater than, or equal to, a pressure threshold value and high-level duration threshold value, respectively. The controller may be configured to control the air source to provide a further one of the series of high-level PAP in response to the pressure level and the high-level duration of a current one of the series of steps of the high-level PAP being sensed to have a pressure and a duration that is not greater than, or equal to, a pressure threshold value and the high-level duration threshold value, respectively. The controller may be configured to increase the duration of each of the series of high-level PAP by a predetermined period of time. The controller may be configured to increase the pressure of each of the series of high-level PAP by a predetermined pressure.

In accordance with embodiments of the present system, there is also disclosed a method of controlling a mechanical insufflation-exsufflation (MI-E) device. The method may include acts of controlling an air source to deliver at least one mechanically assisted cough (MAC) to a patient in response to at least one of a target breathing flow and a target inhalation time period being sensed by at least one sensor, and controlling the air source to deliver a series of high-level PAP, when the at least one of a target breathing flow and a target inhalation time period is not sensed by the at least one sensor. Each in the series of high-level PAP provided over a high-level duration being followed by a low-level PAP provided over a low-level duration. The series of high-level PAP increasing in at least one of a pressure level and a duration from a prior one in the series of high-level PAP. Each in the series of high-level PAP starting in response to a corresponding inhalation trigger. The series of low-level PAP each having a pressure level that is lower than the pressure level of each in the series of high-level PAP. The method, wherein the act of controlling the air source to deliver at least one mechanically assisted cough (MAC) to the patient in response to at least one of the target breathing flow and the target inhalation time period being sensed by the at least one sensor may include an act of at least one of increasing the high-level duration of each of the series of high-level PAP by a predetermined period of time and increasing the pressure of each of the series of high-level PAP by a predetermined pressure.

### BRIEF DESCRIPTION OF THE DRAWINGS

It should be expressly understood that the drawings are included for illustrative purposes and do not represent the scope of the present system. It is to be understood that the figures may not be drawn to scale. Further, the relation between objects in a figure may not be to scale and may in fact have a reverse relationship as to size. The figures are intended to bring understanding and clarity to the structure of each object shown, and thus, some features may be exaggerated in order to illustrate a specific feature of a structure. In the accompanying drawings, like reference numbers in different drawings may designate identical or similar elements, portions of similar elements and/or elements with similar functionality. The present system is explained in further detail, and by way of example, with reference to the accompanying drawings which show features of various exemplary embodiments that may be combinable and/or severable wherein:

FIG. 1 is a graph illustrating corresponding flow and pressure waveforms over time which show good patient-device synchrony;

FIG. 2 which is a graph illustrating corresponding flow and pressure waveforms over time which show good patient-device cooperation;

FIG. 3 is a graph illustrating corresponding flow and pressure waveforms over time showing patient-device asynchrony;

FIG. 4 is a graph illustrating corresponding flow and pressure waveforms over time showing patient-device asynchrony;

FIG. 5 is a graph illustrating corresponding flow and pressure waveforms over time showing patient-device asynchrony;

FIG. 6 is an illustrative functional flow diagram performed by a process in accordance with embodiments of the present system;

FIG. 7 is an illustrative functional flow diagram performed by a process in accordance with embodiments of the present system;

FIG. 8 shows is a portion of a graph illustrating the P-V curve showing a relationship between the pressure and volume of the gas within the gas flow path in accordance with embodiments of the present system;

FIG. 9 shows a portion of a graph illustrating a gas pressure waveform within the gas flow path in accordance with embodiments of the present system;

FIG. 10 shows a graph illustrating flow and pressure waveforms as a function of time in accordance with embodiments of the present system;

FIG. 11 is an illustrative front planar view of a vest including an FTD in accordance with embodiments of the present system;

FIG. 12 shows a graph illustrating a flow, pressure, and force waveforms as a function of time for various steps in accordance with embodiments of the present system; and

FIG. 13 shows a block diagram of a portion of a system in accordance with embodiments of the present system.

### DETAILED DESCRIPTION OF THE PRESENT SYSTEM

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well-known devices, circuits, tools, techniques, and methods are omitted so as not to obscure the description of the present system.

The term and/or and formatives thereof should be understood to mean that only one or more of: the recited elements may need to be suitably present (e.g., only one recited element is present, two of the recited elements may be present, etc., up to all of the recited elements may be present) in a system in accordance with the claims recitation and in accordance with one or more embodiments of the present system. In the context of the present embodiments, the terms “about”, “substantially” and “approximately” denote an interval of accuracy that a person skilled in the art will understand to still ensure the technical effect of the feature in question which in some cases may also denote “within engineering tolerances”. The term may indicate a deviation from the indicated numerical value of ±20%, ±15%, ±10%, ±5%, ±1% ±0.5% or ±0.1%.

As used herein, the term patient, patients, or formatives thereof may be interchangeably used with the terms subject, subjects, user, and/or users unless the context indicates otherwise. Further, the term operator of embodiments may be referred to as operator or clinician unless the context indicates otherwise.

As used herein, the term breath pacing is intended to convey a controlled sequence of high and low level positive airway pressure delivered to a patient in response to a patient initiated inhalation that encourages over the sequence for the patient to have an inhalation that satisfies at least one of a target breathing flow, pressure and/or a target inhalation time period.

It should be appreciated that embodiments of the present system may provide enhanced device-patient synchrony during MI-E therapy based upon passive timing delivery of mechanical insufflation using breath pacing which may be ideal for users (e.g., subjects, patients, etc.) who, for example, may be incapable of following instructions and/or may otherwise not follow instructions during the performance of MI-E therapy such as infants, the disabled, and the like.

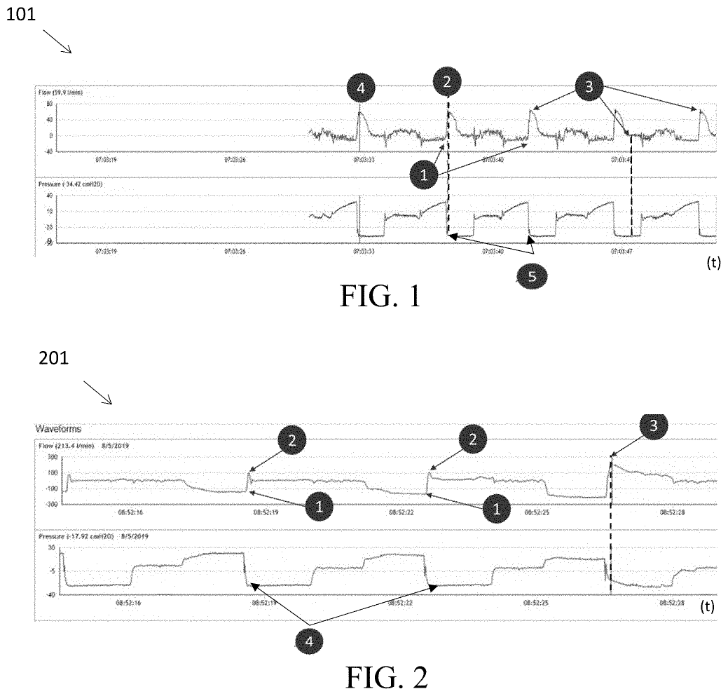

Methods which provide improved MI-E device synchrony with patient breathing based on passively monitoring flow and pressure waveforms during breathing cycles of patients to trigger the delivery of mechanical insufflation at a time when these patients are not holding their breath will now be described below with reference to FIGS. 1 through 13 and the corresponding text.

Several examples illustrating when effective MI-E therapy may be provided in contrast with therapy delivery conditions that may provide ineffective therapy delivery in the pediatric NMD patient population are illustrated with reference to FIGS. 1 through 5. These figures are in a form of graphs obtained for MI-E therapy delivery for patients that are either able or not able to follow commands and, thus, illustrate characteristics that may be utilized to to provide MI-E therapy delivery in accordance with embodiments of the present system. In each of the following graphs, points-of-interest (POIs) will be illustrated as numerals within circles and may be referred to as points.

FIG. 1 is a graph 101 illustrating corresponding flow and pressure waveforms over time which show good patient-device synchrony thereby enabling effective MI-E therapy delivery. In this example, the patient appears to be cooperating with MI-E therapy with a good mask seal illustrated by flow decay to baseline as illustrated at 1. Patient-device synchrony is present with an absence of a decompression spike as illustrated at 2. Expiratory flow is present as illustrated by a flow that lasts three-quarters through an exsufflation phase as illustrated at 3. The peak cough flow (PCF) is about sixty liters-per-minute (lpm) A target inhale pressure (P_{I}) is a pressure level that is associated with effective cough therapy. Clinical consensus is that a target inhale pressure setting for MI-E therapy should be set ≥40 cm H₂O, though in some cases a clinician might set it to a lower setting. As readily appreciated, peak cough flow (PCF) is considered a flow rate associated with clinically effective airway clearance index of expectoration of airway secretions in subjects. Peak cough flow may be measured by a peak flow meter that measures how fast a user may push air out of their lungs when blowing out as hard and as fast as they can. The peak flow measures how open the airways are in the lungs. For example, effective peak cough flow in healthy subjects may exceed values of 360 to 400 L/min. A typical inspiration before coughing reaches 80 to 90% of vital capacity (VC). Vital Capacity (VC) is the total amount of air exhaled after maximal inhalation. The value may be about 4800 mL for a given patient and it varies according to age and body size. It is calculated by summing tidal volume (TV), inspiratory reserve volume (IRV), and expiratory reserve volume (ERV).

VC=TV+IRV+ERV

In the instance indicated in FIG. 1, effective MI-E therapy may be performed during exhalation, such as at the start of exsufflation/exhalation as illustratively shown at any of points 5 since pressure and inhale time thresholds may be detected to have been met previously in accordance with the present system as discussed in more detail below. As is clear from the flow and pressure diagrams of FIG. 1, that patient flow appears to have ended expiration before the set exsufflation time had ended. This would indicate the exhalation time may be too long and may be shortened to coincide with the end of patient flow. Also, in this example, the PCF of approximately 60 lpm, would likely be increased if one were to synchronize a chest or abdominal thrust at the start of the exsufflation phase/start of patient exhaled flow as described further herein. In this way in accordance with the present system, a mechanically assisted cough may be presented during some portion of exhalation following confirmation that the pressure and/or inhale time thresholds are detected to have been met. For example, once the breath had been captured or paced and the inhale pressure and time thresholds are met, the mechanically assisted cough may be synchronized to the next patient triggered (initiated) breath. In other words, once the time and pressure thresholds are met, the next patient effort may be used as the mechanism to introduce a mechanically assisted cough that is comprised of positive pressure delivered during the inhalation phase for a set period of time (e.g., such as the length of time of the patient initiate inhalation), followed by (e.g., immediately) negative pressure delivered during the exsufflation phase for a set period of time (e.g., such as for length of time of patients exhalation. Patient exhaled flow or peak cough flow (PCF) are metrics that may be for evaluating how effectively the MAC was introduced/accepted by the patient. An optimum MAC delivery window may be determined by meeting both the inhale time and inhale pressure thresholds. For example, the inhale time threshold may be preset by the clinician (e.g. 1.5 seconds), or may be determined by the longest inhale time period achieved with active patient flow that is greater or equal to, for example, 1 second. The inhale pressure threshold may be preset (e.g. 40 cm H₂O) or may be determined by the upper inflection point in the pressure volume curve as discussed in more detail herein. Alternatively, the pressure threshold may be determined by the highest positive pressure achieved that was tolerated by the patient (associated with inhale flow as opposed to breath holding), for example, that is greater or equal to 30 cm H₂O.

Flow and pressure waveforms for MI-E therapy performed on a nine-year-old patient is shown in FIG. 2 which is a graph 201 illustrating corresponding flow and pressure waveforms over time which show good patient-device cooperation and therefore illustrate conditions upon which effective MI-E therapy may be performed during exhalation in accordance with embodiments of the present system. In this example, it is noted that a mask leak may be observed at least during a period leading up to an including point 1. Accordingly, a seal between the patient and a mask preferably should be enhanced to reduce leakage prior to providing a MAC in accordance with the present system. Little exhale flow is present at a first machine-assisted-cough (MAC) as illustrated at point 2. Passive exhalation on a third MAC is illustrated at point 3.

In the instance indicated in FIG. 2, effective MI-E therapy may be performed during exhalation such as illustratively shown at any of points 4 since pressure and inhale time thresholds may be detected to have been met previously in accordance with the present system as discussed in more detail below. In this way in accordance with the present system, a mechanically assisted cough may be presented when the optimum MAC window has been determined and will be synchronized to the next patient trigger following confirmation that the pressure and inhale time thresholds are detected to have been met.

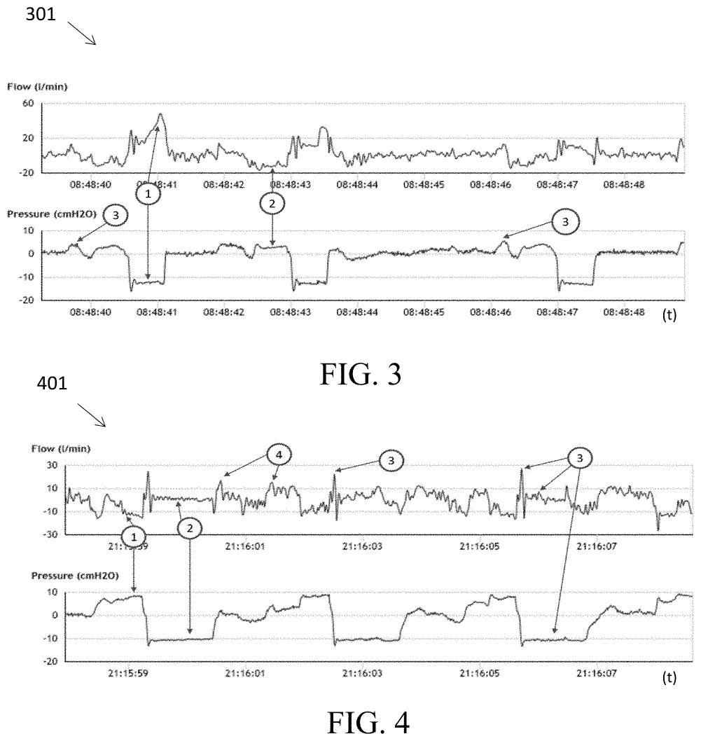

Several examples showing patient profiles of pediatric patients that are either too young or otherwise unable to follow commands for any reason (e.g., due to cognitive limitations) are illustrated with reference to FIGS. 3 through 5. As is shown, these patient profiles indicate noticeable asynchrony between the MI-E device and the patient's breathing pattern, which may result in ineffective MI-E therapy delivery.

FIG. 3 is a graph 301 illustrating corresponding flow and pressure waveforms over time showing patient-device asynchrony. In the present graph 301, the patient was seven months old and suffered from spinal muscle atrophy (SMA). As shown, the highest observed PEFR is less than 60 lpm, and passive exhalation is evidenced by a lack of counter pressure at point 1. At point 2, the patient does not appear to be accepting the breath and a target inhale pressure of 20 cm (centimeters) H₂O is not reached. Point 3 illustrates patient-device asynchrony as patient exhalation is seen during a pause phase of the cycle. As such, synchrony can be attempted to be achieved through breath pacing in accordance with embodiments of the present system as described herein.

FIG. 4 is a graph 401 illustrating corresponding flow and pressure waveforms over time showing patient-device asynchrony. In the present embodiment, the patient was five months old and suffered from SMA. At point 1, positive pressure is not being accepted during insufflation, and the CoughAssist MI-E device is not reaching the target inhale pressure setting. At point 2, no expiratory flow is noted during the negative pressure exsufflation phase. At point 3, an oropharyngeal decompression spike is seen with no active effort being applied by the patient, no passive expiratory flow noted, and no PCF rate is greater than 30 lpm. As such, synchrony can be attempted to be achieved in accordance with embodiments of the present system as described herein further.

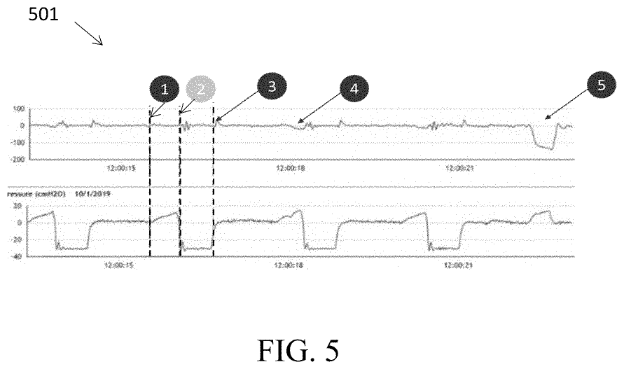

FIG. 5 is a graph 501 illustrating corresponding flow and pressure waveforms over time showing patient-device asynchrony. In the present embodiment, the patient was an infant. At point 1, the patient appears to be holding their breath as there appears to be no flow response on inhale phase of an inhalation exhalation cycle. At point 2, no exhale flow is present further indicating that the patient is holding their breath. At point 3, a small exhalation during a pause phase is illustrated. At point 4, a small inhalation is seen but this inhalation does not yield any measurable PCF. At point 4 a large leak on positive pressure on an inhale breath is seen. As such, synchrony can be attempted to be achieved in accordance with embodiments of the present system as described below.

In summary, the preceding figures show that when a patient, such as an infant/child, is too young or unable to follow commands, several problems may be encountered leading to ineffective MI-E therapy in the absence of the system in accordance with the present system. The present system may address problems associated with patients that exhibit one or more problems, thereby providing an opportunity to bring about patient-device synchrony such that effective MI-E therapy may be delivered. Problems that may be detected in accordance with the present system may include one or more of failure to accept insufflation, patient—device asynchrony (e.g., breath holding, patient exhalation during the pause phase), no exhale flow, low Peak Cough Flow (PCF) rates, and/or no or low effective cough volume (ECV).

As discussed, the present system and method improves MI-E device synchrony with patient breathing based on passively monitoring the flow and pressure waveforms during breathing cycles of the patient to trigger a delivery of a mechanically assisted cough (MAC) at a time when the patient is not holding their breath, such as during an exhalation following a favorable inhalation by the patient.

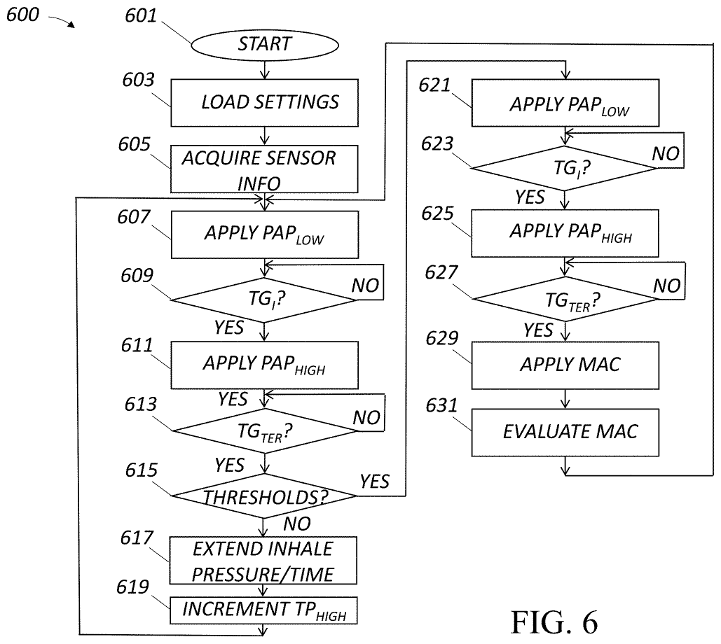

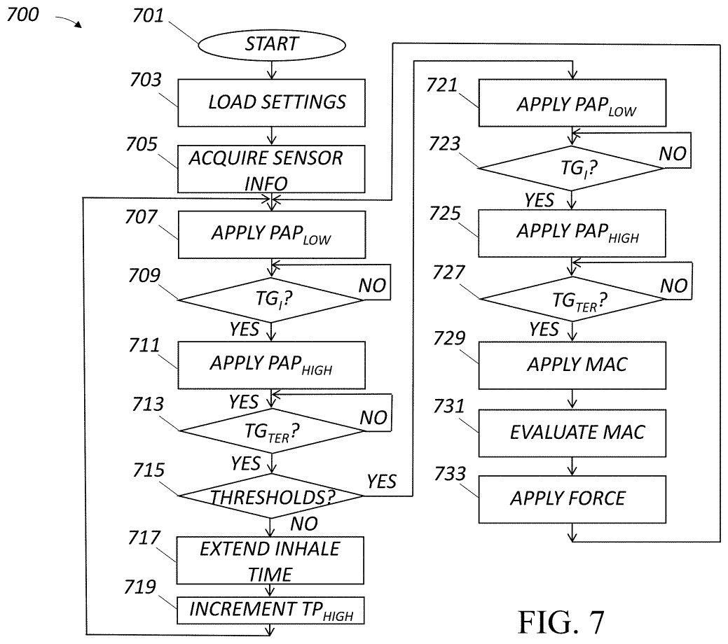

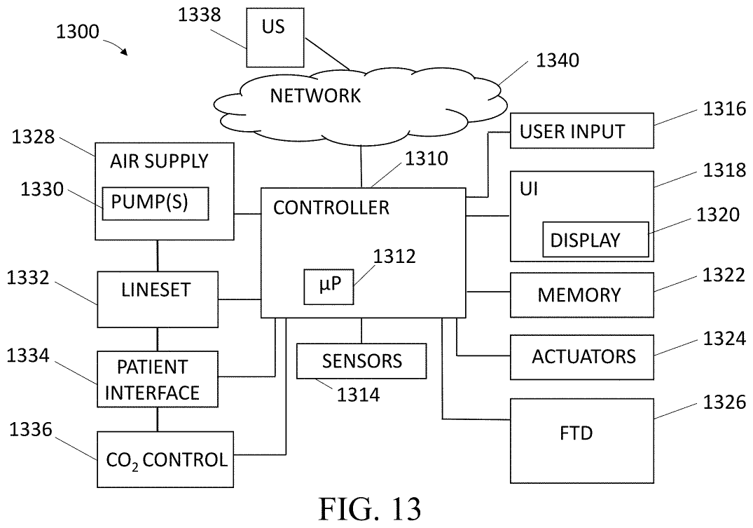

FIG. 6 is an illustration 600 which shows a functional flow diagram performed by a process in accordance with embodiments of the present system. FIG. 7 is an illustration 700 which shows a functional flow diagram performed by a further process in accordance with embodiments of the present system. The processes 600, 700 may be performed using one or more processors, computers, controllers, etc., (hereinafter each of which may be referred to as the controller for the sake of clarity) communicating over a network and may obtain information from, and/or store information to, one or more memories which may be local and/or remote from each other (e.g., see, FIG. 13, controller 1310 and memory 1322). The process 600, 700 may include one of more of the following acts. In accordance with embodiments of the present system, the acts of process 600, 700 may be performed using a controller operating (e.g., programmed, constructed) in accordance with embodiments of the present system. Further, one or more of these acts may be combined and/or separated into sub-acts, as may be desired. Further, one or more of these acts may be skipped or the order between acts may be changed depending upon system settings operating in accordance with embodiments of the present system. With reference to process 600, in operation, the process may start during act 601 and then proceed to act 603.

During act 603, the process may initialize the one or more settings that may be input by an operator in real time or may be obtained from a memory of the system. Such information may be stored in a memory of the system as system information (SI). The settings may, for example, include information related to initialization and/or default settings such as threshold settings, pressure settings, step settings, user settings, etc. These settings may be formed, set, updated, and/or reset by the system and/or operator and stored as a portion of the SI in the memory. Accordingly, the controller may obtain the SI from a memory of the system and/or may render information requesting input by the operator of one or more settings, depending upon system settings. After completing act 603, the process may continue to act 605.

During act 605, the process may obtain sensor information from the one or more sensors of the system, such as pressure sensors, flow sensors, temperature sensors, humidity sensors, gas analysis sensors. Each of these sensors may monitor corresponding parameters, form corresponding information, and provide this information to a breath monitoring unit (BMU) of a controller of the system (e.g., a program portion that may be executed by the controller) for further processing. For example, it is envisioned that the pressure sensors may monitor pressure, ambient pressure, etc., may form corresponding pressure information, and provide this pressure information to the controller for further processing. Similarly, it is envisioned that the flow sensors may monitor fluid flow (e.g., as of respiratory gasses) in one or more locations such as in a line-set (LS) or a patient interface (PI), may form corresponding flow information, and provide this flow information to the controller for further processing.

The gas analysis sensors may be configured to determine a concentration of one or more types of gasses within a sampled airflow such as Carbon dioxide (CO₂), Oxygen (O₂), etc., form corresponding sensor information (e.g., CO₂ concentration in percent) and provide this information to the controller for further processing. The controller may identify each sensor directly or via the sensor information provided by each of the sensors (e.g., which may include information identifying the type of information, and/or sensor which formed it, etc.). The process may obtain the sensor information in real time, and/or on a periodic or non-periodic intervals depending upon system settings. The BMU and/or other portion of the controller may process the sensor information and generate corresponding pressure and/or waveform information (e.g., pressure and waveforms, respectively, for one or more locations throughout the system, e.g., such as within a patient line-set (LS) or other patient interface (PI)). The BMU may monitor flow and pressure waveforms in accordance with embodiments of the present system. The LS may be coupled to the PI and at least some of the flow and/or pressure sensors may provide information related to flow and/or pressure within a gas flow path (GFP) of one or more of the LS and/or PI.

Thus, for the sake of clarity, pressure, flow, and volume may refer to pressure, flow, and volume, respectively, of a gas flow within the GFP of one or more of the LS and/or a PI coupled thereto. The PI may include a one-way or check valve that may help prevent buildup of CO₂, which may be passively or actively controlled (e.g., by the controller) through the valves or other systems as described further herein. After competing act 605, the process may continue to act 607.

During act 607, the process may be operative (e.g., programmed) to apply low-level positive airway pressure (PAP_{LOW}) to the patient until the patient initiates a spontaneous breath. Accordingly, the process may be operative to control one or more pumps and/or pressure regulators of the system to provide a pressurized flow of gas (e.g., air) within the GFP. This pressurized flow of gas may have (absent other forces such as a breath of a patient) a positive pressure (e.g., at the PI) which may correspond to a threshold low pressure value (TP_{LOW}). The TP_{LOW} may depend upon system settings, may be operator defined (e.g., 4 cm H₂O), may be preset (e.g., as stored in the SI), and/or may be determined by the system (e.g., in real time).

As discussed, a low-level PAP (PAP_{LOW}) is initially delivered as a baseline pressure. The baseline PAP level may be user-defined (e.g., 4-10 cm H₂O such as 4 cm H₂O) as discussed or may be based on the pressure level that creates a lower inflection point (LIP) in a pressure volume (P-V) curve as will be discussed below (e.g., 8 cm H₂O). A PAP value that is set to the LIP may offer additional therapeutic value to a pediatric patient using MI-E therapy. Closing capacity is the volume in the lungs that is associated with small airway and alveolar closing. Closing capacity is the point during expiration when small airways begin to close. This phenomenon may be seen in pediatric patients <6 years of age that have a functional residual capacity (FRC) below closing capacity. FRC is the volume remaining in the lungs after a normal, passive exhalation.

The present system may provide advantages in an embodiment using a dynamic algorithm for determination of the baseline PAP setting using the LIP include: (1) increasing FRC above closing capacity, (2) mitigating small airway and alveolar collapse, (3) ultimately improving lung compliance, (4) aiding in both pressure habituation and breath-pacing to create an optimal MI-E delivery window. By the term dynamic algorithm, it is intended to convey a mathematical optimization method and a computer programming method.

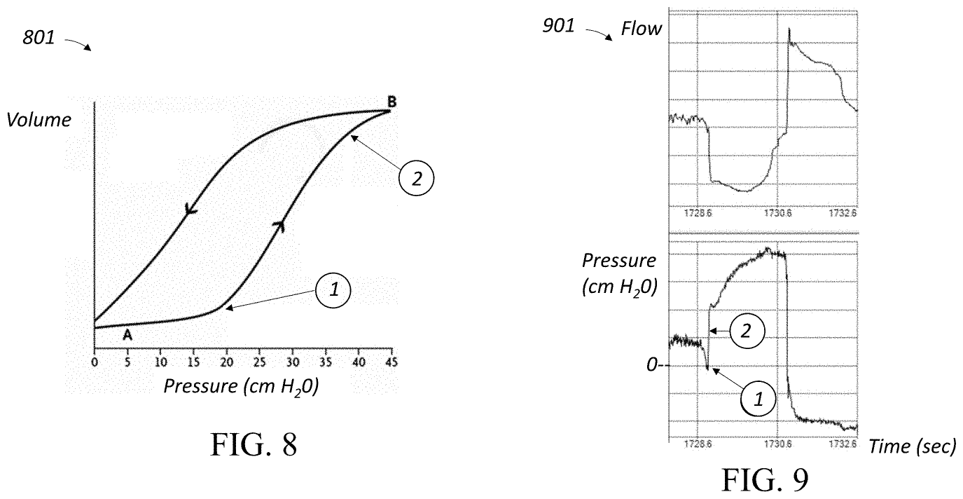

For example, during operation the TP_{LOW} may be based upon a pressure of gas at a detected lower inflection point (LIP) of a pressure volume (P-V) curve as will be discussed below. For example, to determine the value of TP_{LOW}, the process may monitor sensor information relating to a P-V curve for a given patient as set forth in FIG. 8.

FIG. 8 shows a portion of a graph 801 illustrating a given patient's P-V (pressure verses volume) curve. The graph 801 of FIG. 8 illustrates a relationship between the pressure and volume of gas within the gas flow path of a patient in accordance with embodiments of the present system. Point 1 illustrates the LIP which is an inflection point wherein there is a sudden change in the volume of the gas in the gas flow path along a lower leg of the P-V curve. This inflection point in the P-V curve may be detected by signal processing software of the controller. In some embodiments, the LIP may be detected when it is determined that a tangent to the P-V curve has occurred and/or may have a rate-of-change (ROC) which may be greater than a threshold ROC_{I} (TROC) for an inhalation cycle. An upper inflection point (UIP) 2 may also be detected by the process in a similar manner and will be discussed below. Depending upon system settings, once the LIP is determined, the process may set TP_{LOW} to this value or to another value based on the determined LIP (e.g., 10% above or below the determined LIP and may apply the PAP_{LOW} to the LS accordingly (e.g., see, FIG. 6, acts 605, 607), such as until the patient initiates a spontaneous breath. In some embodiments, a triggered breath may be generated that is volume targeted and pressure limited. A patient initiated inhalation effort results in a drop in both device measured pressure and flow. This change in pressure or flow can be used to “trigger” or initiate the inhale positive pressure (insufflation) or initiate the MAC. Volume targeted may be achieved by a clinician entering a target volume (e.g. 500 mL) which may be used as a threshold to determine when pressure delivery should be terminated (e.g., when the entered volume is achieved). A maximum pressure setting may be used to limit the total pressure delivered when the breath is volume targeted. For example, a volume target could be 300 mL, however the machine delivered positive pressure may still be limited to a maximum pressure such as 35 cm H₂O. This triggered breath may then be used to detect the LIP and ultimately determine the baseline PAP level (e.g., the PAP_{LOW}) that may be delivered during act 607 (e.g., may be delivered during a pause phase of MI-E). In this way, the patient is triggering, that is to say initiating the machine delivered MAC such as a positive pressure, by the patient initiated breath. After completing act 607, the process may continue to act 609.

During act 609, the process may determine whether an inhalation trigger (TG_{I}) is detected. The inhalation trigger is a sensor reading received by the processor that is indicative of the patient starting a spontaneous breath (e.g., a detected change in pressure, flow, etc., within the within the LS, PI, etc.). The inhalation trigger indicates that the patient has initiated a spontaneous breath as may be indicated by a small drop in flow rate and also may be detectable in the flow waveform as previously discussed.

Accordingly, when the inhalation trigger (TG_{I}) is detected, the process may continue to act 611. However, when the inhalation trigger (TG_{I}) is not detected, the process may repeat act 609 and may continue to apply PAP_{LOW} to the LS. For example, the process (e.g., through operation of a suitably programmed processor) may determine that the inhalation trigger (TG_{I}) is detected (e.g., detection of a spontaneous breath by the patient) by using one or more methods in accordance with settings of the system. For example, in one method the process may detect the inhalation trigger (TG_{I}) in response to sensing a drop in pressure and/or sensing negative pressure within the GFP which may occur in response to a spontaneous breath of the patient.

FIG. 9 shows a portion of a graph 901 illustrating a detailed graph of a gas flow and pressure waveform within the gas flow path in accordance with embodiments of the present system. In yet another embodiment, the process may detect the inhalation trigger (TG_{I}) in response to detecting that pressure within the GFP has fallen below atmospheric pressure. As shown, a mechanically assisted cough at point 2 is initiated or triggered by the patient briefly “pulling” the pressure below ambient during start of inhalation at point 1. This may be referred to as a negative pressure trigger. As the flow diagram demonstrates, reciprocal flow occurs as the patient inhales during the positive pressure delivery during the insufflation phase. The pressure that is delivered may be set by the clinician or algorithmically determined such as by the upper inflection point on the pressure volume curve. When the pressure of the gas in the GFP is less than 0 (e.g., negative) as shown at point 1 of FIG. 9, the process may determine that the inhalation trigger (TG_{I}) is detected. This may be indicative of the patient beginning to inhale. In yet other embodiments, the process may analyze the P-V curve and may detect the inhalation trigger (TG_{I}) in response to a change in the pressure waveform. In yet other embodiments, the process may detect the inhalation trigger (TG_{I}) when it is determined that the pressure is less than a threshold value such as a low-pressure threshold (LPT). Accordingly, the process may compare, the current pressure (as shown in the pressure waveform) to the LPT to determine whether the current pressure is less than or equal to the LPT. In the affirmative, the process may determine that the inhalation trigger (TG_{I}) is detected. In the negative, the process may determine that the inhalation trigger (TG_{I}) has not yet been detected. The LPT may be set by the system and/or user.

Referring to FIG. 6, during act 611, the process may be operative to apply high-level positive airway pressure (PAP_{HIGH}) to the LS. Accordingly, the process may control one or more pumps and/or pressure regulators of the system to provide a pressurized flow of gas (e.g., air) to the GFP at PAP_{HIGH} which is at a greater pressure than PAP_{LOW}. This pressurized flow may be at a positive insufflation pressure and may correspond to a threshold high pressure value (TP_{HIGH}) for the current cycle. In accordance with embodiments, TP_{HIGH} may be set by the clinician, may be determined by the upper inflection point in the pressure volume curve, may be determined by the pressure to achieve a target volume, etc. A normal range may be 30-50 cm H₂O, although a clinician may also set a higher maximum pressure ceiling (e.g., 70 cm H₂O). TP_{HIGH} may be increased with each inhalation cycle until thresholds are met as will be described below with reference to act 617. This transition in pressure from PAP_{LOW} to PAP_{HIGH} may occur just after detection of the spontaneous inhalation which causes the pressure to momentary become negative (e.g., see, point 2 of FIG. 9) which may signify the inhalation trigger (TG_{I}). Thus, after detection of the inhalation trigger (e.g., signifying the patient's spontaneous inhalation, as signified by 2 of FIG. 9, the process provides a flow of gas at a higher pressure than PAP_{LOW} so as to transition from PAP_{LOW} to PAP_{HIGH} within the GFP.

Upon detecting the inhalation trigger (TG_{I}) (e.g., indicative of the start of an inhalation), the process may also start an inhalation timer which may count a current inhalation time (T_{I}) which corresponds to a total inhalation time during which the patient inhales (e.g., spontaneously) for the current cycle.

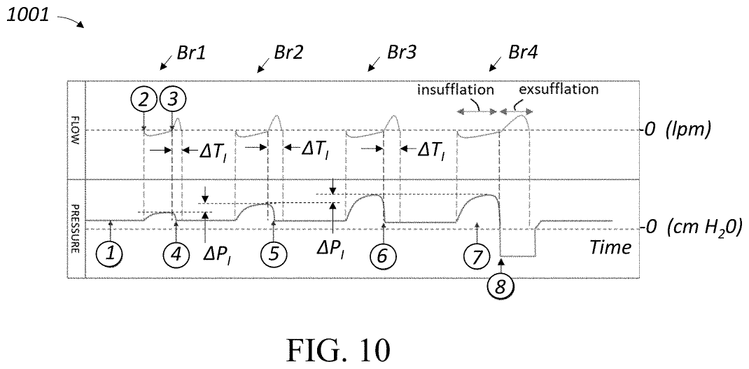

Flow and pressure waveform will now be discussed with reference to FIG. 10 which shows a graph 1001 illustrating a flow and pressure waveforms as a function of time in accordance with embodiments of the present system. The graph 1001 illustrates a series (e.g., a sequence of) of breaths (Br1 through Br4) each with inhalation and exsufflation phases as illustrated with reference to Br4, though present for each of Br1 through Br4 as discussed below.

Referring to FIG. 10, the process may apply PAP_{LOW}, which is a positive insufflation pressure, at point 1 until the inhalation trigger (TG_{I}) is detected (e.g., detection of a spontaneous inhalation) at which time the process may increase pressure to PAP_{HIGH} as illustrated at point 2 where an insufflation flow is indicated.

Thus, once an inhalation trigger is detected, embodiments of the present system may deliver an increase in positive airway pressure until the patient terminates their breath (e.g., end of inhalation/insufflation] as determined by a flow decay and zero flow crossing at point 3. The present system will synchronize an increase in the baseline PAP level until the patient terminates their breath as for example determined by a flow cycle threshold (peak flow decay or zero flow crossing). The pressure increase, which is graphically illustrated in FIG. 10 below, may be determined by the clinician (e.g. 4 cm H₂O), may be determined as a percentage of the user-defined pressure target (e.g. PAP_{HIGH}=40 cm H₂O, pressure step=10% or 4 cm H₂O), or may be determined as a percentage of the LIP (e.g., LIP=10 cm H₂O, pressure step=60% of the LIP or 6 cm H₂O). PAP_{HIGH} is a maximum pressure threshold value and may be used for determined when to deliver a MAC as discussed.

In accordance with embodiments of the present system, the inhalation trigger (TG_{I}) may be detected in response to detection of a spontaneous breath such as through detection of a negative pressure waveform and/or a change in flow characteristics (e.g., see point 1 of FIG. 9) either of which may occur when the patient begins to inhale.

Referring back to FIG. 6, after completing act 611, the process may continue to act 613 where the process may determine whether a termination trigger (TG_{TER}) is detected. Accordingly, when the exhalation trigger (TG_{TER}) is detected, the process may continue to act 615. However, if the termination trigger (TG_{TER}) is not detected, the process may repeat act 613 and may continue to apply the PAP_{HIGH} to the LS. Generally, the process may determine that the termination trigger (TG_{TER}) is detected in response to the patient terminating the current breath as may be determined by a flow decay and/or a zero flow crossing (e.g., see at point 3 of FIG. 10 where the patient terminates their breath (i.e., has finished inhaling), resulting in the system determining an end of insufflation. For example, in one embodiment, the process may analyze the pressure volume curve to determine whether an upper inflection point (UIP) may be detected, may analyze the pressure waveform to determine whether a pressure peak is detected and/or may detect a change in flow/pressure to detect that the patient has begun to exhale (e.g., see point 3 of FIG. 10). The upper inflection point would be conceptually used to determine the positive pressure setting to be used during the insufflation phase when a mechanically assisted cough is delivered to the patient.

Referring back to FIG. 6, during act 615 the process may determine whether the current inhalation time (T_{I}) is greater than or equal to an inhalation time threshold value (ITT) and/or the current TP_{HIGH} is equal to or greater than the maximum pressure threshold value (PAP_{HIGH}). In the affirmative, the process may continue to act 621 (where the next synchronized breath will introduce a single, mechanically assisted cough (MAC) and in the negative the process may continue to act 617. Although two limitations are set forth above, in some embodiments only one of these conditions may be used depending upon system and/or user settings.

With regard to the pressure threshold value (PAP_{HIGH}), depending upon system settings, this value may be a preset value set by a user or system (e.g., 35 cm H₂O, etc.) or may be based on an arbitrary pressure below the upper inflection point (UIP) on the Pressure-Volume curve (e.g., see, FIG. 8, point 2). For example, the clinician could set a fixed pressure reduction to be used along with the upper inflection point to determine the inhale pressure setting that is used during the insufflation phase of the mechanically assisted cough. For example, the clinician may have a pressure reduction range choice of 0-5 cm H₂O, clinician selects 2 cm H₂O, upper inflection point is determined to occur at 40 cm H₂O, the pressure setting used during the insufflation phase of the MAC would be 38 cm H₂O (40-2). A series of pressure stepped breaths over a predetermined time period or an automatically determined time period may be performed until the UIP is accurately determined. For example, a 1^{st} patient effort may result in 5 cm H₂O delivery during the inhalation phase. During a next patient effort, the PAP level may increase to 10 cm H₂O, while the subsequent PAP level may increase to 15 cm H₂O, etc. As readily appreciated, a different step size may be used.

With regard to the inhalation time threshold value (ITT), depending upon system settings this value of ITT may be a preset value set by a user or system (e.g., 1 second, etc.), may be derived from a longest insufflation time, for example, as determined by a zero-flow crossing during the breath pacing phase, and/or may be based upon a calculated inspiratory time constant (TC_{I}) of the respiratory system. For example, a measurement of dynamic compliance and airways resistance may be utilized for performing the inhalation time constant calculation: TC_{I}=C_{DYN}×R_{AW}×3 (95% of inspiratory capacity) to ensure adequate lung filling and alveolar equilibration time. Airways resistance or R_{AW} may be determined by a modified forced oscillation technique pressure application during an ideal measurement window. For example, airway resistance may be determined by a forced oscillation technique (i.e., PAP value oscillated according to a set Hz). Compliance may be determined by dividing the tidal volume by the plateau pressure minus PEEP. Settings for the maximum pressure threshold value (PAP_{HIGH}) and the inhalation time threshold value (ITT) may be stored in the SI for later use.

During act 617, the process may extend the current insufflation time (T_{I}) (e.g., which is an inhalation time during which PAP_{HIGH} is applied) by a current period ΔT_{I} as may be set by the system and/or user (e.g., see, ΔT_{I}, FIG. 10). For example, the present system may extend the inhale time (e.g., extend insufflation) to a calculated point past the patient's own breath termination as determined by either a flow cycle threshold or zero flow crossing. Further, the increase in insufflation time may be calculated as a percentage increase over the last inhale time period/insufflation time period, as measured or as determined by using a predetermined time step such as 0.1 or 0.2 seconds.

The process may also increment the period ΔT_{I} by some other desired value such that with each inhalation, the period of insufflation ΔT_{I} may increase. Thus, a next value of ΔT_{I} may equal the current value of ΔT_{I} plus (+) a given or variable increase. Thus, with each successive inhalation, the process may extend the total inhalation time (T_{I}) during which PAP_{HIGH} is applied by a current value of ΔT_{I}. Thus, during each successive inhalation, PAP_{HIGH} may be applied for an increasing period of time when compared with the time period of a previous inhalation as shown in FIG. 10 until a target inhalation period is detected (see, FIG. 10, point 7 as described below). Thus, embodiments of the present system may extend an inhale time (insufflation) for a period past the patient's spontaneous inhalation termination.

A transient inspiratory hold when achieved, may be used to calculate a static lung compliance C_{STAT} value which may in turn be used to replace a C_{DYN} value in an inhale time constant calculation to improve accuracy when acts 607, 609, 611, 613, 615, 617, 619 are repeated following act 619 as described, otherwise a quasi-static (inhale flow <10 L/sec) lung compliance estimate may be used to determine C_{DYN} as noted in the description of act 615 above.

Thereafter, the process may continue to act 619. During act 619, the process may increase the value of TP_{HIGH} by a threshold value such as a ΔP_{I} (e.g., see, FIG. 10) which may be set by the system and/or operator and stored in a memory of the system such as in the SI. Thus, a next value of TP_{HIGH} may be equal to the current value of TP_{HIGH}+ΔP_{I}. Accordingly, during (and following) each successive inhalation, the PAP_{HIGH} during insufflation may be increased by ΔP_{I} and/or ΔT_{I} of insufflation may be increased as shown in FIG. 10. After completing act 619, the process may repeat act 607.

In accordance with embodiments of the present system, acts 607 through 619 may form a portion of a breath pacing phase and may be repeated for each breath with patient-initiated, device-modulated increases (after the first breath of the sequence) in inhale pressure (TP_{HIGH}) and/or insufflation time for each breath (e.g., see, points 4 5, 6, for first through third breaths (Br1 through Br3, shown in FIG. 10), respectively), until the pressure and inhale time thresholds for an optimum MI-E delivery window are met (e.g., see, point 6, FIG. 10). The gradual increase in PAP_{HIGH} and/or T_{I} extension are designed to capture and drive or pace the patient's breathing to provide conditions for an effective delivery of a mechanically assisted cough (MAC). After extending the inhale time T_{I} and/or increases in PAP_{HIGH} during acts 617, 619, the process may cycle back to act 607 where the baseline PAP level is applied. In accordance with embodiments, the baseline PAP level may also be adjusted following a breath such as during a pause (e.g., increase following one or more breaths).

Acts 621 through 627 may be similar to acts 607 through 613, respectively, though occur following detection of thresholds during act 615. Accordingly, only a brief description of these acts will be provided for the sake of clarity and reference will be made to acts 607 through 613 for further detail.

During act 621, the process may be operative to apply low-level positive airway pressure (PAP_{LOW}). At this time, pressure and inhalation period thresholds for the delivery of an optimized MAC window (e.g., see, point 8, FIG. 10) have been met and a final insufflation will be delivered prior to delivering a MAC as will be discussed below. The optimum MAC window (MAC_{OW}) may be determined by reaching one or both of pressure (e.g., PAP_{HIGH}) and length of time (e.g., TI_{MAX}) thresholds. In accordance with embodiments, PAP_{HIGH} (e.g., the maximum pressure setting or level) may be user defined (e.g., 40 cm H₂O) or may be set by the upper inflection point (UIP). For example, the PAP_{HIGH} may be set at the UIP or at a percentage, such as 10 or 20 percent above or below the UIP For example, if the UIP corresponds to 50 cm H₂O, 10% higher would equal a positive pressure setting of 55 cm H₂O, a 10% lower would equal a setting of 45 cm H₂O, etc. TI_{MAX} may also be user-defined (e.g., 1 second), derived from the longest insufflation time, or may be determined by zero flow crossing achieved during the breath pacing phase (e.g., 1.3 seconds). TI_{MAX} may also be based on a calculated inspiratory time constant of the respiratory system. The inspiratory time constant is the time required for inflation up to 63% of the final lung volume. For example, inspiratory flow per second=(L/min/60 seconds) Raw=(PIP−Pplat) Time Constant=(Raw/flow L/cm H₂O), wherein PIP is peak airway pressure and Pplat is the patient plateau pressure.

After completing act 621, the process may continue to act 623. During act 623, the process may determine whether an inhalation trigger (TG_{I}) is detected. Accordingly, when the inhalation trigger (TG_{I}) is detected, the process may continue to act 625. However, when the inhalation trigger (TG_{I}) is not detected, the process may repeat act 623 and may continue to apply PAP_{LOW} to the LS.

During act 625, the process may be operative to apply high-level positive airway pressure (PAP_{HIGH}) to the LS, for example at the same pressure as the previous inhalation as the threshold high pressure value (TP_{HIGH}) may not have been increased for the current inhalation. After completing act 625, the process may continue to act 627 where it may determine whether a termination trigger (TG_{TER}) is detected. Accordingly, when the termination trigger (TG_{TER}) is detected, the process may continue to act 629. However, when the termination trigger (TG_{TER}) is not detected, the process may repeat act 627 and may continue to apply the PAP_{HIGH} to the LS.

During act 629, the process may begin an exsufflation wherein it may apply a mechanically assisted cough (MAC) to the LS. Accordingly, the process may be operative to control one or more pumps and/or pressure regulators of the system to provide a vacuum (e.g., negative pressure) within the GFP suitable for the MAC. This is illustrated with reference to FIG. 10 at point 8 where the process delivers a single MAC (in the illustrative embodiment as a negative pressure during exsufflation however may also be provided in a form such as an abdominal and/or chest thrust during the exsufflation phase directly following an insufflation phase). In the present embodiments, only a single MAC is provided during exsufflation. In yet other embodiments more than one MAC may be performed. In case of a vacuum being applied as the MAC, the level of vacuum utilized (e.g., the negative pressure setting), may depend upon system settings, may be system or operator defined and/or may be stored in the SI for later use. For example, the level of vacuum utilized may be from −30 to −50 cm H₂O as for example set by any of the above noted processes. Thereafter, the process may, depending upon system settings, update system settings such as the SI in accordance with embodiments of the system. Referring back to FIG. 6, after competing act 629, the process may continue to act 631.

During act 631 the process may evaluate the MAC for effectiveness (ECV or PCF) based on the acquired flow and pressure waveforms, before the process repeats starting at act 607 i.e., returns to the breath pacing phase until the next optimal MAC window is identified. The MAC optimal window may be determined as the combination of meeting the inhale time threshold and inhale pressure thresholds. For example: the patient's breath may be synchronized (no asynchrony, patient accepting the positive pressure) to the inhale pressure threshold of 40 cm H₂O or more for an inhale time duration threshold of 1.5 seconds or more. The system may return to breath pacing for a minimum of one patient-triggered breath to evaluate for the presence of MAC_{OW} for determining whether a subsequent MAC may be delivered.

In accordance with embodiments of the present system, a MAC may be introduced without or following breath pacing as described with reference to FIGS. 1 and 2, when proper conditions for the introduction of the MAC (e.g., during patient-initiated inhalation(s) and/or exhalation(s)) are detected (e.g., see 5 and 4 in FIGS. 1 and 2, respectively).

In accordance with an embodiment, synchronized delivery of M I-E volume mode may be performed with patient breathing. In this embodiment, once the patient inhale effort is detected (e.g., trigger event), the present system may deliver a constant flow rate until the volume reaches the pre-determined inhale volume. The flow rate may be adjusted to optimize the inhale time or the patient comfort. The volume step may be determined by the clinician (e.g. 200 ml increment), as a percentage of the user-defined volume target (e.g. Vti_max=2 L, volume step=10% or 200 ml), or as a percentage ideal body weight or other PFT parameters (i.e., VC, TLC, etc.) (e.g. 15 kg ped patient with 6 ml/kg target tidal volume, 90 ml volume increment).

Thereafter, the present system may extend the inhale time to a preset inhale time. Alternatively, the inhale time may be adjusted automatically to achieve the target volume. For example, a static compliance and resistance calculation may be made as the plateau pressure and zero flow is reached during each inhale cycle.

For example, C_stat=Target Volume/(Peak inhale pressure−PEEP)

Res_stst=(Peak inhale pressure−PEEP)/flow rate.

As before, the acts may be repeated with patient-initiated, device modulated increases in inhale volume and inhale time. Thereafter, the MAC_{OW} may be determined by reaching one or both of a volume and time target value. For example, the volume threshold value may be user defined or may be equal to UIP. The UIP may be identified through a single breath when the target volume is sufficiently large enough to reach the true UIP.

As before, when the target volume and/or target time are achieved, a MAC may thereafter be provided to the patient.

In some embodiments, the process may render information on a user interface of the system, such as to provide information to an operator of a current operating state of the system, such as whether the system is pacing breaths and/or delivering MACs. It is further envisioned that the process may end at any time if desired.

Accordingly, the process in accordance with embodiments of the present system, may provide for improved device-patient synchrony during MI-E therapy using breath pacing.

Embodiments of the present system may be provided with a patient interface including a one-way check valve that may provide for the venting of carbon dioxide from the GFP during use thus enhancing patient safety. This may reduce or entirely prevent issues due to build-up of carbon dioxide in the GFP during the breath pacing acts which may last multiple breathing cycles (e.g., 4-10 breath cycles) in some embodiments.

In an embodiment, flushing of CO₂ build up in the MI-E circuit may be accomplished with transient negative pressure delivery synchronized with the patient's own breath termination. The frequency, duration, and negative pressure level (e.g., −3 cm H₂O for 0.5 seconds) may be applied during synchronized, transient negative pressure applications and may be determined by the number and length of the patient-initiated breaths between MI-E applications For example, a bench model may be setup to see how many breaths at a given tidal volume result in a high CO₂ build up in a 6-foot breathing circuit. Conversely, a negative pressure model may then be constructed to determine what negative pressure needs to be delivered and for how long to safely flush the CO₂ build-up. For example, a low negative pressure setting of −3 to −5 cm H₂O for a short period of time (e.g., (0.1-0.3 sec)) may be sufficient to flush CO₂ build up.

It is envisioned that some embodiments may be fitted with a carbon dioxide exhaust line which may be flow coupled to the GFP to introduce a low-level negative pressure, e.g. −3 cm H₂O which may be triggered by an exhalation by the patient. A low-level negative pressure application has been shown to be an effective CO₂ flushing methodology in bench tests.

In yet another embodiment, a compensation port (e.g., a leak port, an exhalation port, a passive valve, an actively controlled valve, etc.) may be flow coupled to the MI-E circuit to reduce or entirely eliminate CO₂ buildup (beyond ambient levels) within the MI-E circuit by venting CO₂ and introducing fresh air. The controller may include a leak/pressure compensation algorithm to compensate for pressure leaks and/or other changes in pressure, flow etc., which may actively control one or more portions of the system such as the compensation port to reduce or eliminate CO₂ buildup within flow circuits of the system such as within the patient interface, etc., while having little or no impact upon pressure delivery. For example, the controller may control the pressure compensation valve to exhaust CO₂ from the patient interface.

Synchronized Abdominal Chest Thrust

In some embodiments, after the patient accepts the positive pressure, insufflated breath successfully, MI-E therapy may be enhanced by providing a synchronized abdominal chest thrust (e.g., to the chest of the patient) under the control of the controller. This may be beneficial in cases where it is determined that the patient produces low peak cough flow (PCF) rate or low effective cough volume (ECV). Accordingly, the process may identify the low PCF or low ECV. The setting for low PCF could be a clinician determined threshold or preset, for example <160 lpm. Effective cough volume could also be set by the clinician, or the threshold automatically determined as a percentage of the inhaled tidal volume. For example, if the VT were 1 liter and the ECV threshold was set at 50%, then any ECV less than 500 mL would be considered a suboptimal mechanically assisted cough. In accordance with embodiments, the system may generate an indication(s) and may render the indication(s) on a rendering device of the system such that an operator of the system may be informed that a synchronized abdominal chest thrust will be applied and/or is suggested (e.g., for the operator to apply including suggested timing) to enhance therapy. This synchronized abdominal chest thrust may be performed by the system during an optional window that may occur prior to, following or during a time when the MAC is performed or may a force transmitting device (FTD) action may be provided in place of or together with a MAC For example, a synchronized abdominal thrust or chest thrust may be be synchronized to the start of exsufflation with the presence of patient exhaled flow to be effective.

For example, embodiments illustrating synchronized abdominal chest thrust during exsufflation following successful insufflation will now be discussed with reference to FIG. 7 and FIG. 10 in which acts 701 through 731 may be similar to acts 601 through 631, respectively, of the process 600 of FIG. 6. Accordingly, the description of FIG. 6 and the corresponding waveform diagram of FIG. 10 may be referred to for further detail. With reference to FIG. 7, following act 731, the process may continue to act 733 where the process may provide a synchronized chest thrust, for example, at the start of the MAC window (e.g., see, point 8, FIG. 10). With reference to FIG. 7, upon applying the MAC (e.g., see point 8, FIG. 10) and/or evaluating the MAC (see, acts 729 and 731, the system may be operative to activate a force transmitting device (FTD) to apply a force to the chest or abdomen of the patient when the MAC is applied. Thereafter, the system may control the FTD to release this force before beginning the next inspiratory period. After completing act 733, the process may continue to act 707 to repeat acts or may simply return to act 727 to evaluate the inhalation/insufflation to determine whether a further MAC/force to the chest or abdomen of the patient may be repeated.

Mechanical chest or abdominal thrusts may be desirable where the patient is not able to cooperate with a synchronized cough effort during the exsufflation phase. Chest and abdominal thrusts may be employed to improve peak cough flow rates (PCFs) rates during MI-E therapy. Increasing the PCFs as well as the effective cough volume (ECV) during the exsufflation phase of MI-E therapy delivery is achieved by triggering automated abdominal and/or chest thrusts that are synchronized with the start of the exsufflation phase following the end of the insufflation phase (e.g., see, point 8, FIG. 10). A threshold for delivering the synchronized thrust may include achieving an effective insufflation phase (as described above with reference to the process 600).

The automated chest or abdominal thrust may be a viable means of raising the PCFs/ECV in patients that are unable to cooperate, follow commands and/or respond to breath pacing adequately. An increase in PCFs/ECV would improve airway clearance for this therapeutic modality.

Accordingly, in embodiments of the present system a force transmitting device (FTD) such as a belt, wrap, a vest, etc., may be worn by the patient around the patient's chest and may be activated by a controller of the system such as at the initiation of the MAC or other time in accordance with embodiments of the present system. The FTD may be coupled to a controller of the system and may include one or more bladders, an electro-active polymer (EAP) actuator, a mechanically operated tensioning device, etc., that may be operated by the controller to increase pressure at one or more times as may be determined by the controller to transfer a force effective to perform a chest or abdominal thrust upon the patient. The FTD may include one or more actuators and/or one or more sensors, such as force sensors, that may sense a force subject thereupon, form corresponding sensor information, and provide this sensor information to a controller of the system for further processing, such as to determine a force applied to the abdomen or chest of the patient. Indication of the forces may be rendered by the controller on a rendering device of the system.

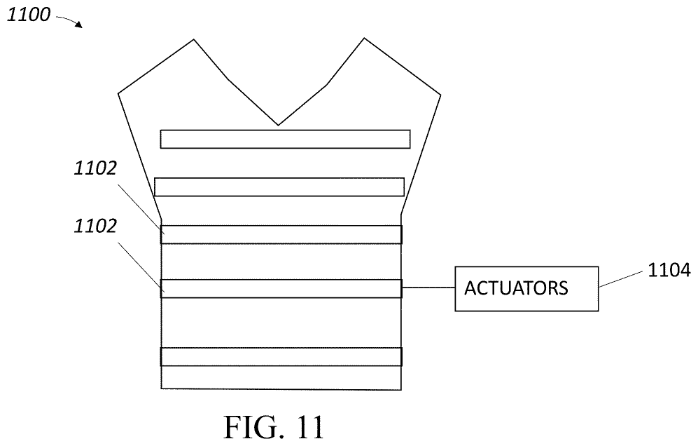

FIG. 11 is an illustration which shows a front planar view of a vest 1101 including one or more FTDs 1102 in accordance with embodiments of the present system. The FTD(s) 1102 may extend around the vest 1101 and may include one or more pneumatic or hydraulic tubes, bands, pads, etc., (generally bands) which may include chambers configured to receive a fluid under pressure which may cause the FTD 1102 to expand and generate a force under the control of a controller of the system. Actuators 1104 may be coupled to the bands 1102 and may be configured to control the flow of fluid (e.g., gas or liquid) to the bands 1102 to control expansion or contraction of the corresponding one or more bands 1102.

In yet other embodiments, the FTD 1102 may include bands formed from an EAP polymer or polymers which may expand or contract under the control of the controller to generate a force. These bands may be coupled to an actuator which, in conjunction with the system controller, may control an electrical voltage applied to the bands to cause them to expand and/or contract under the control of the controller. Accordingly, the FTD 1102 may be operative, under the control of the controller and/or operator, to selectively generate desired forces so as to provide for an automated or manual chest or abdominal thrust as may be desired. The rear pan view of the vest 1101 may be similar to that shown in FIG. 11.

Mode Saliency to Operator

It is envisioned that some embodiments of the present system may provide or otherwise render salient audio-visual indication(s) of the particular mode of operation in which the system is in, i.e., breath pacing, mechanical insufflation, or exsufflation. Such an indication may provide the operator with useful information of an operating mode of the system which may ensure not only that device operation is safe but also that MI-E therapy is effectively delivered without interruption. Since it may take several breathing cycles (e.g., 4-10 breath cycles) before the pressure and inhale time thresholds are attained to allow mechanical insufflation, the operator may be uncertain when the MAC has been delivered, which may lead to unexpected interruption or termination of therapy when, for example, the operator may prematurely decide to terminate operation of the system. Accordingly, it is envisioned that embodiments of the present system may be operative to detect an operating state of the system and whether there are any errors, whether the system has completed therapy successfully, etc., and render on a rendering device of the system an indicator of such. For example, during therapy the system may render an indication, such as one or more solid colored light(s) to indicate to the operator to continue providing therapy (e.g., therapy continuing). When the therapy has been completed and may be terminated, the system may render a solid light to indicate when to terminate therapy (e.g., such as at the end of therapy). It is also envisioned that the system may render information indicating therapy status such as a solid green light (e.g., emitted by a light-emitting diode (LED)) which may indicate for the duration of the therapy that the therapy is going well. When sufficient and successful therapy has been provided, it is envisioned that the system may detect this and render an indication of such using lighting, for example, a blinking green light (e.g., a green LED) to alert the operator that the therapy was successful and may be terminated. When, for example, it is detected that the therapy is not going well, the system may detect this and render an indicator of such displaying, for example, a continuous red LED light, followed by a blinking red LED light and error tone in case this situation persists for more than 2 cycles. In other embodiments status indicators may be rendered on a display screen of the system such as a touchscreen display for interaction with a user.

Actively Synchronized Delivery of Mechanical Insufflation with Patient Breathing

It is also envisioned that embodiments of the present system may include enhanced device-patient synchronization. This may be beneficial in situations in which breath pacing approach may be unsuccessful due to extreme patient agitation and/or persistent or erratic breath holding. Accordingly, embodiments of the present system may include methods which may actively trigger a patient breath (i.e., initiate inspiration) hold which can then be used to support synchronized delivery of MI-E therapy.

A reflex known as the Hering-Breuer deflation reflex (aka the excito-inspiratory reflex) may serve to shorten exhalation when the lung is deflated and may be initiated by embodiments of the present system. For example, this reflex may be initiated either by stimulation of stretch receptors or stimulation of proprioceptors activated by lung deflation. This reflex may also be triggered using, for example, chest compressions as described by S. Hannam, D. M. Ingram, S. Rabe-Hesketh, and A. D. Milnerin Characterisation of the Hering-Breuer deflation reflex in the human neonate. Respir Physiol 2001; 124(1):51-64, incorporated herein as if set out in its entirety. Chest compressions may be provided using any suitable FTD such as the FTD 1102 operating in accordance with embodiments of the present system. For example, any suitable FTD such as the FTD 1102 or the like, may be selectively activated by a controller of the system to trigger a patient breath through the application of the automatic application of chest compressions/thrusts. The FTD may include any suitable device such as a vest or wrap that may be activated using any suitable activation method or methods as described with reference to the FTD 1120. Accordingly, the FTD may be selectively operative to deliver abdominal and/or chest thrusts during exsufflation. In this case mechanical insufflation will be synchronized with the chest compressions/thrusts as presented in the sequence of acts depicted in FIG. 8. It is important to highlight that in this case the breaths are not patient (e.g., infant) initiated but rather are device initiated via the application of chest compression/thrusts. The chest compressions/thrusts may be applied to the chest of the patient once or a plurality of times, such as repeatedly until the patient terminates the breath.

It is envisioned that the force applied during the chest compressions/thrusts may be gradually increased in steps until conditions are met for insufflation such as described above regarding an increasing in fixed steps as described above, without exceeding a maximum safe force. The SI may include information related to maximum force to be applied to the patient by one or more of size, age, etc. The process may then obtain this information to determine a maximum safe force to be applied and may obtain sensory feedback from one or more force sensors of the system to determine that the applied force (as sensed) does not exceed the maximum safe force for the patient. For example, two-finger and two-thumb chest compression during neonatal resuscitation suggests that peak chest compression forces in the range 35-50 N may be safe for infants as described by Dellimore K., Heunis S., Gohier F., et al., in Development Of A Diagnostic Glove For Measurement Of Chest Compression Force And Depth During Neonatal CPR published in Proc 35th Annual International IEEE EMBS Conference of the IEEE Engineering in Medicine and Biology Society, Osaka, Japan, 2013:350-353. Incorporated herein as if set out in entirety. This approach may be applied in combination with the breath pacing approach described above with reference to the embodiments of the present system (e.g., see embodiment of FIG. 6) until a suitable pressure threshold and inhale time threshold are met.

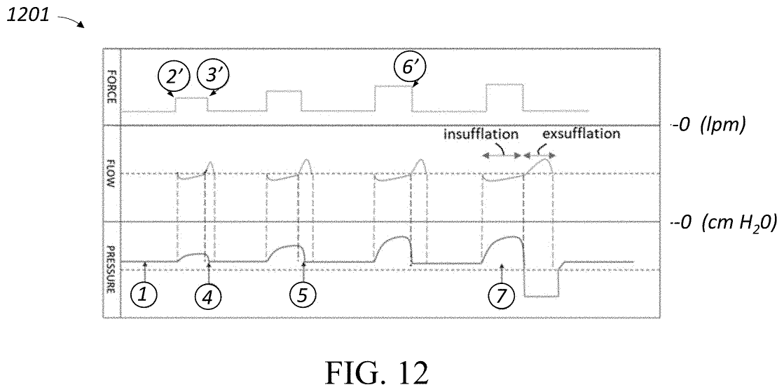

FIG. 12 shows a graph 1201 illustrating a flow, pressure, and force waveforms as a function of time for various acts in accordance with embodiments of the present system. The flow, pressure, and force waveforms of illustrate various acts performed by embodiments of the present system which may be operative to enhance device-patient synchrony. Low-level PAP (e.g., see, point 1) may be applied until a breath is triggered by application of a chest compression/thrust (e.g., see, point 2′), which in turn triggers an increase in device pressure until the patient terminates its breath (e.g., see, point 3′)—the device extends inhale time for a predetermined time period (e.g., milliseconds past the zero-flow crossing, see, point 4) [until cycling back to low-level PAP. It should be noted that as in other embodiments, the low-level PAP may be different levels during subsequent cycles or may have two or more different levels that repeat during a cycle or in subsequent cycles.

This process repeats with a chest thrust/compression-initiated, device modulated increases in inhale pressure and time (e.g., see, point 5), until the pressure and inhale time thresholds are met (e.g., see, point 6′). The next synchronized breath may be utilized to introduce a single, mechanically assisted cough (e.g., see, point 7 and following).

Embodiments of the present system may be applied in domestic and clinical settings to deliver a more optimal and effective MI-E therapy to patients who are too young or unable to follow commands, such as infants or individuals who have learning disabilities than conventional systems. This therapy is specifically relevant to patients with neuromuscular disorders such as spinal muscular atrophy, Duchenne muscular dystrophy, polymyositis, hereditary spinal ataxia, and the like. It should also be appreciated that the present system may be utilized to provide automated MI-E therapy to patients who are cooperative in that the system may make the determination (e.g., automatically) of when conditions and/or timing are optimal or not. In this way, even a patient that exhibits satisfactory patient-system synchrony, may benefit by the proper timing and delivery of MI-E therapy without needing intervention from a clinician.

Further, embodiments of the present system may provide systems that are operative to trigger mechanical insufflation based on breath pacing and are operative to overcome problems such as those that may be caused by breath holding or dishabituation to the negative and positive pressure sensation that may be produced by the MI-E therapy.

FIG. 13 shows a block diagram of a portion of a system 1300 in accordance with embodiments of the present system. The system 1300 may include one or more of: a controller 1310, sensors 1314, a user input interface 1316, a user interface (UI) 1318, a memory 1322, actuators 1334, an FTD 1326, an air supply 1328, a line set (e.g., a patient line set) 1332, a patient interface 1334, a CO₂ control 1336, a network 1340, and an optional user station (US) 1338 each of which may be coupled to and/or communicate with each other using any communication method or methods such as wired, optical, flow, and/or wireless communication methods. The system 1300 may be operative under the control of the controller 1310.

The US 1338 may include any suitable user station such as a smart phone or the like that may be configured to communicate with other portions of the system 1300 such as the controller 1310 via any suitable communication method or methods such as via the network 1340.

The controller 1310 may include one or more logic devices such as a microprocessor (P) 1312 and may control the overall operation of the system 1300. A more detailed description of the controller 1310 may be given below. It would be appreciated that in some embodiments the controller 950 may include digital and/or analog control circuitry.

For example, one or more portions of the present system such as the controller 1310 may be operationally coupled to the memory 1322, the user interface (UI) 1318 including a rendering device such as the display 1320, the sensors 1314, and the user input interface 1316, the actuators 1324, the FTD 1326, the air supply 1328, the line set 1332, the patient interface 1334, the CO₂ control 1336, the network 1340, and the US 1338.

The memory 1322 may be any type of device for storing application data as well as other data related to the described operation such as the SI, patient information, etc. The application data and other data may be received by the controller 1310 for configuring (e.g., programming) the controller 1310 to perform operation acts in accordance with the present system. The controller 1310 so configured becomes a special purpose machine particularly suited for performing in accordance with embodiments of the present system. For example, the controller 1310 may be configured to perform or coordinate/calculate/determine as described with FIGS. 6 and 7. In being so programmed, the present system and controller overcomes many problems in prior systems that are not suitable for delivering MI-E therapy when breathing from a patient is erratic and/or when the time for delivery is optimal. Prior systems relied on skilled clinicians to speculate when an airway clearing procedure may be performed such as described by Chatwin M., Ross E., Hart N., Nickol A H, Polkey M I, Simonds A K in Cough Augmentation With Mechanical Insufflation/Exsufflation In Patients With Neuromuscular Weakness, published in Eur Respir J 2003; 21(3):502-508 and Ishikawa Y., Bach J R, Komaroff E., Miura T., Jackson-Parekh R in Cough Augmentation In Duchenne Muscular Dystrophy, published in Am J Phys Med Rehabil 2008; 87(9):726-730, each of which is incorporated herein as if set out in entirety. Other system relied on fixed timing for delivery of the MAC regardless of the state of the patient. However, due to the limitations of the prior technology, in many instances, sufficient MI-E therapy could not previously be administered to large groups of patients.

In accordance with the present system, the controller 1310 may render content, such as still or video information, on a rendering device of the system such as on the display 1320 of the UI 1318. This information may include information related to operating parameters, instructions, timing, feedback, and/or other information related to an operation of the system or portions thereof. For example, the controller 1310 may receive sensor information from one or more of: the sensors 1314 and compare airflow information related to current airflow, pressure, gas composition (e.g., O₂, CO₂ percentages, etc.), force (e.g., chest force, etc.) through one or more portions of the system (e.g., such as through the CO₂ control 1336, the line set 1332, the patient interface 1334, and/or the air supply 1328) with threshold airflow information, threshold pressure information, threshold gas composition information, and/or threshold force information, respectively, and control the system accordingly. In this way, the controller 1310 may determine whether the valves, pumps, actuators, etc., is/are operating outside of operating parameters and may render results of this determination for the convenience of a user. The controller 1310 may include a breath monitoring unit which may monitor the breathing of the patient by acquiring flow and pressure information (e.g., flow and pressure signals) from the sensors. The controller 1310 may then process the acquired flow and pressure signals to regulate inhale pressure, inhale time, and/or delivery of mechanical insufflation by the system.

The sensors 1314 may be situated at one or more portions of the system and may sense related parameters, form corresponding sensor information, and provide this sensor information to the controller 1310 for further processing. For example, the sensors 1314 may include sensors such as flow, pressure, force, gas analysis, etc., which may form corresponding sensor information (e.g., gas flow, gas pressure, etc.) and provide this information to the controller 1310 for further analysis. The sensors 1314 may distributed throughout the system.

The user input interface 1316 may include a keyboard, a mouse, a trackball, or other device, such as a touch-sensitive display, which may be stand alone or part of a system, such as part of a MAC device, a laptop, a personal digital assistant (PDA), a mobile phone (e.g., a smart phone), a smart watch, an e-reader, a monitor, a smart or dumb terminal or other device for communicating with the controller 1310 via any operable link such as a wired and/or wireless communication link. The user input interface 1316 may be operable for interacting with the controller 1310 including enabling interaction within a UI 1318 as described herein. Clearly the controller 1310, the sensors 1314, the user input interface 1316, the user interface (UI) 1318, the memory 1322, the actuators 1334, the FTD 1326, the air supply 1328, the line set (e.g., a patient line set) 1332, the patient interface 1334, the CO₂ control 1336, the network 1340, and the optional a user station (US) 1338 may all or partly be a portion of a computer system or other device such as MAC device, etc.

The UI may be operative to provide audio/visual feedback to the operator of the present system and may inform the operator of operating parameters, operating states, etc. For example, the UI may render information indicative of when a mechanically assisted cough has been successfully delivered.

The methods of the present system are particularly suited to be carried out by a computer software program, such program containing modules corresponding to one or more of: the individual acts or acts described and/or envisioned by the present system. Such program may of course be embodied in a computer-readable medium, such as an integrated chip, a peripheral device or memory, such as the memory 1332 or other memory coupled to the controller 1310.

The program and/or program portions contained in the memory 1322 may configure the controller 1310 to implement the methods, operational acts, and functions disclosed herein. The memories may be distributed, for example between the clients and/or servers, or local, and the controller 1310, where additional processors may be provided, may also be distributed or may be singular. The memories may be implemented as electrical, magnetic or optical memory, or any combination of these or other types of storage devices. Moreover, the term “memory” should be construed broadly enough to encompass any information able to be read from or written to an address in an addressable space accessible by the controller 1310 (e.g., by the P 1312). With this definition, information accessible through a network such as the network 1340 is still within the memory, for instance, because the controller 1310 may retrieve the information from the network 1340 for operation in accordance with embodiments of the present system.

The controller 1310 is operable for providing control signals and/or performing operations in response to input signals from the user input device 1316 as well as in response to other devices of a network, such as the sensors 1314 and executing instructions stored in the memory 1322. The P 1312 may include one or more of: a microprocessor, an application-specific and/or general-use integrated circuit(s), a logic device, etc. Further, the P 1312 may be a dedicated processor for performing in accordance with the present system and/or may be a general-purpose processor wherein only one of many functions operates for performing in accordance with the present system. The P 1312 may operate utilizing a program portion, multiple program segments, and/or may be a hardware device utilizing a dedicated or multi-purpose integrated circuit.

The actuators 1324 may, under control of the controller 1310, control one or more valves, pumps, motors, and/or the FTD 1326. For example, the actuators 1324 may include one or more valve actuators that may control the pressure and/or flow of a fluid such as air used by the system such as in the line set 1332.

The CO₂ control may include one or more ports (e.g., balance valves, etc.) or valves (e.g., active and/or passive) that may be operative to control the buildup of CO₂ in one or more portions of the system such as in one or more of the line-set 1332 and the patient interface 1334.

The FTD 1326 may include any suitable device such as a vest or wrap that may be activated using any suitable activation method or methods as described with reference to the FTD 1120. Accordingly, the FTD may be selectively operative to deliver abdominal and/or chest thrusts to the patient during one or more operating stages such as during exsufflation.

The air supply 1328 may include one or more fans and/or pumps 1330 that may be configured to supply a flow of air suitable for performing PAP therapy in accordance with embodiments of the present system.

The line set 1332 may couple the patient interface 1334 to the air supply 1328 and may include any suitable air flow path or paths such as may be provided by one or more tubes, hoses, or the like. The line set 1332 may further include one or more sensors, filters, and/or valves.

The patient interface 1334 may include any suitable patient interface such as a mask, a trach, and/or mouthpiece configured to be coupled to the line set 1332. When coupled together, the patient interface 1334 and the line set 1332 may form one or more of a mask circuit, a trachea circuit, and/or a mouthpiece circuit. The patient interface 1334 may form a non-invasive patient interface. The patient interface 1334 may include a one-way check valve operative to reduce or entirely prevent the build-up of CO₂ in the flow portions of the systems such as the patient interface 1334. This valve may be a part of or independent from the CO₂ control 1336.

The CO₂ control 1336 may be coupled to one or more of the air supply 1328, the line set 1332, and the patient interface 1334 and may be configured to control the buildup of CO₂ gas within one or more of the of the air supply 1328, the line set 1332, and the patient interface 1334. The CO₂ control 1336 may be passive (e.g., a one-way valve permitting the outflow of exhaled gasses from the patient interface 1334) or may be actively controlled by the controller 1310 in accordance with embodiments of the present system.

The controller 1310 may be operable to control one or more ventilation devices and/or other devices as described. Similarly, the controller 1310 may be operable to control peripheral devices operating for example with a PAP device, pressure, humidification, flow, force, and heating circuits operating in accordance with embodiments of the present system.

Accordingly, embodiments of the present system may provide a system to monitor the state of the ventilator and/or provide a user interface for the user to control settings and/or parameters of the ventilator using a local and/or remote communication. A wireless communication link such as a Bluetooth™ or Wi-Fi™ link between portions of the ventilator and the rendering device 138 and the system may enable rendering of system parameters on a UI of the rendering device 138 which may also provide an entry area in which a user may change parameters such as ventilator settings, parameters, etc., of the system. Additionally, this link may be configured to link two or more PAP and/or MAC systems using a two-way connection. With this connection, battery system parameters may be rendered and/or airflow rates, pressure, temperature, humidity, gas analysis, etc., may be displayed and/or adjusted by the user. Parameters such as temperature, pressures, voltages, battery charge, specific oxygen (SpO₂), carbon dioxide (CO₂), humidity, force, current operating state, etc., may be determined and rendered on a rendering device of the system such as on the display 1320 of the system for the convenience of the user. Through the UI 1318, the user may interact to select and/or change parameters in accordance with embodiments of the present system.

Further variations of the present system would readily occur to a person of ordinary skill in the art and are encompassed by the following claims.

Embodiments of the present system may also be operative with continuous positive airway pressure (CPAP)/bilevel positive airway pressure (BiPAP) devices and other positive airway pressure devices operating in accordance embodiments of the present system.

Finally, the above discussion is intended to be merely illustrative of the present system and should not be construed as limiting the appended claims to any particular embodiment or group of embodiments. Thus, while the present system has been described with reference to exemplary embodiments, it should also be appreciated that numerous modifications and alternative embodiments may be devised by those having ordinary skill in the art including using features that are described with regard to a given embodiment with other envisioned embodiments without departing from the broader and intended spirit and scope of the present system as set forth in the claims that follow. In addition, any section headings included herein are intended to facilitate a review but are not intended to limit the scope of the present system. In addition, the specification and drawings are to be regarded in an illustrative manner and are not intended to limit the scope of the appended claims.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function;
e) any of the disclosed elements may be comprised of hardware portions (e.g., including discrete and integrated electronic circuitry), software portions (e.g., computer programming), and any combination thereof,
f) hardware portions may be comprised of one or both of analog and digital portions;
g) any of the disclosed devices, features and/or portions thereof may be combined together to act in synchronous or sequential timing or separated into further portions unless specifically stated otherwise;
h) no specific sequence of acts or steps is intended to be required unless specifically indicated; and
i) the term “plurality of” an element includes two or more of the claimed elements and does not imply any particular range of number of elements; that is, a plurality of elements may be as few as two elements and may include an immeasurable number of elements.

What is claimed is:

1. A mechanical insufflation-exsufflation (MI-E) device, comprising:
an air source configured to provide patient airway pressure including positive airway pressure (PAP);
a patient interface coupled to the air source and configured to be flow coupled to a user;
at least one sensor configured to sense air pressure and flow at the patient interface; and
a controller coupled to the air source and the at least one sensor, the controller being configured to control the air source to deliver at least one mechanically assisted cough (MAC) to the patient in response to at least one of a target breathing flow and a target inhalation time period being sensed by the at least one sensor, and when the at least one of a target breathing flow and a target inhalation time period is not sensed by the at least one sensor, the controller is configured to control the air source to deliver a series of high-level PAP, each in the series of high-level PAP provided over a high-level duration being followed by a low-level PAP provided over a low-level duration, the series of high-level PAP increasing in at least one of a pressure level and the high-level duration from a prior one in the series of high-level PAP, and each in the series of high-level PAP starting in response to a corresponding inhalation trigger, wherein the series of low-level PAP each having a pressure level that is lower than the pressure level of each in the series of high-level PAP.

2. The MI-E device of claim 1, further comprising a force transmitting device (FTD) coupled to the controller, wherein the controller is configured to control the FTD to apply an abdominal chest thrust that is synchronized to the MAC.

3. The MI-E device of claim 1, wherein the controller is further configured to receive flow and pressure information from the at least one sensor and generate corresponding flow and pressure waveforms.

4. The MI-E device of claim 3, wherein the controller is further configured produce each in the series of high-level PAP in response to detecting an inhalation trigger (TG_{I}) in a form of a change in pressure below ambient in the pressure waveform.

5. The MI-E device of claim 1, further comprising a CO₂ controller coupled to the patient interface and configured to control a concentration of CO₂ gas at the patient interface.

6. The MI-E device of claim 1, wherein the controller is configured to control the air source to deliver the at least one mechanically assisted cough (MAC) to the patient when both of the target breathing flow and the target inhalation time period are sensed by the at least one sensor.

7. The MI-E device of claim 1, wherein the controller is configured to determine whether the pressure level and the high-level duration of a current one of the series of the high-level PAP has a sensed pressure and a duration greater than, or equal to, a pressure threshold value and a high-level duration threshold value, respectively, wherein the controller is configured to control the air source to provide a further one of the series of high-level PAP in response to the pressure level and the high-level duration of a current one of the series of steps of the high-level PAP being sensed to have a pressure and a duration that is not greater than, or equal to, the pressure threshold value and the high-level duration threshold value, respectively.

8. The MI-E device of claim 1, wherein the controller is configured to increase the high-level duration of each of the series of high-level PAP by a predetermined period of time.

9. The MI-E device of claim 1, wherein the controller is configured to increase the pressure of each of the series of high-level PAP by a predetermined pressure.

10. A mechanical insufflation-exsufflation (MI-E) device, comprising:
a controller configured to control an air source to deliver at least one mechanically assisted cough (MAC) to a patient in response to at least one of a target breathing flow and a target inhalation time period being sensed by at least one sensor, and when the at least one of a target breathing flow and a target inhalation time period is not sensed by the at least one sensor, the controller is configured to control the air source to deliver a series of high-level PAP, each in the series of high-level PAP provided over a high-level duration being followed by a low-level PAP provided over a low-level duration, the series of high-level PAP increasing in at least one of a pressure level and the high-level duration from a prior one in the series of high-level PAP, each in the series of high-level PAP starting in response to a corresponding inhalation trigger, wherein the series of low-level PAP each having a pressure level that is lower than the pressure level of each in the series of high-level PAP.

11. The MI-E device of claim 10, wherein the controller is configured to control a force transmitting device (FTD) to apply an abdominal chest thrust that is synchronized to the MAC.

12. The MI-E device of claim 10, wherein the controller is further configured to receive flow and pressure information from the at least one sensor and generate corresponding flow and pressure waveforms.

13. The MI-E device of claim 12, wherein the controller is further configured to produce each in the series of high-level PAP in response to detecting an inhalation trigger (TG_{I}) in a form of a change in pressure below ambient in the pressure waveform.

14. The MI-E device of claim 10, wherein the controller is further configured to control a concentration of CO₂ gas available to the patient.

15. The MI-E device of claim 10, wherein the controller is configured to control the air source (1328) to deliver the at least one mechanically assisted cough (MAC) to the patient when both of the target breathing flow and the target inhalation time period are sensed by the at least one sensor.

16. The MI-E device of claim 10, wherein the controller is configured to determine whether the pressure level and the high-level duration of a current one of the series of the high-level PAP has a sensed pressure and a duration greater than, or equal to, a high-level pressure threshold value and a high-level duration threshold value, respectively, wherein the controller is configured to control the air source to provide a further one of the series of high-level PAP in response to the pressure level and the high-level duration of a current one of the series of steps of the high-level PAP being sensed to have a pressure and a duration that is not greater than, or equal to, the pressure threshold value and the high-level duration threshold value, respectively.

17. The MI-E device of claim 10, wherein the controller is configured to increase the high-level duration of each of the series of high-level PAP by a predetermined period of time.

18. The MI-E device of claim 10, wherein the controller is configured to increase the pressure of each of the series of high-level PAP by a predetermined pressure.

19. A method of controlling a mechanical insufflation-exsufflation (MI-E) device, the method comprising acts of:
controlling an air source to deliver at least one mechanically assisted cough (MAC) to a patient in response to at least one of a target breathing flow and a target inhalation time period being sensed by at least one sensor, and
controlling the air source to deliver a series of high-level PAP, each in the series of high-level PAP provided over a high-level duration being followed by a low-level PAP provided over a low-level duration when the at least one of a target breathing flow and a target inhalation time period is not sensed by the at least one sensor, the series of high-level PAP increasing in at least one of a pressure level and the high-level duration from a prior one in the series of high-level PAP, each in the series of high-level PAP starting in response to a corresponding inhalation trigger, wherein the series of low-level PAP each having a pressure level that is lower than the pressure level of each in the series of high-level PAP.

20. The method of claim 19, wherein the act of controlling the air source to deliver at least one mechanically assisted cough (MAC) to the patient in response to at least one of the target breathing flow and the target inhalation time period being sensed by the at least one sensor comprises an act of at least one of increasing the high-level duration of each of the series of high-level PAP by a predetermined period of time and increasing the pressure of each of the series of high-level PAP by a predetermined pressure.
